(19) United States
(12) Patent Application Publication
(10) Pub. No.: US 2024/0156724 A1
(43) Pub. Date: May 16, 2024

(54) Multi-Dose Container for Ophthalmic Compositions

(71) Applicant: Vyluma Inc., Bridgewater, NJ (US)

(72) Inventors: Kumaresh Soppimath, Skillman, NJ (US); Tushar Hingorani, Bridgewater, NJ (US); Hari A. Attluri, Jersey City, NJ (US)

(73) Assignees: Vyluma Inc., Bridgewater, NJ (US); Vyluma Inc., Bridgewater, NJ (US)

(21) Appl. No.: 18/280,778

(22) PCT Filed: Mar. 14, 2022
(86) PCT No.: PCT/US2022/020246

§ 371 Date: Sep. 7, 2023

Related U.S. Application Data
(60) Provisional application No. 63/161,830, filed on Mar. 16, 2021.

(51) Int. Cl. A61K 9/00; A61K 31/439; A61K 47/32; A61K 47/38

(52) U.S. Cl. CPC A61K 9/0048; A61K 31/439; A61K 47/32; A61K 47/38

(57) ABSTRACT

A sterile multi-dose ophthalmic consumer product, and especially a multi-dose low-concentration atropine eye drop container is disclosed having desirable atropine stability, low loss of viscosity, and low levels of total impurities leached from the container even after extended storage in the container.

This application claims priority to our co-pending US Provisional patent application with the Ser. No. 63/161,830, which was filed Mar. 16, 2021, and which is incorporated by reference herein.

### FIELD OF THE INVENTION

The field of the invention is packaging compositions and methods for ophthalmic formulations, especially as it relates to multi-use containers for eye drop formulations.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Among a large variety of ophthalmic products, eye drops are a common formulation type that enables simple application of a defined quantity of a drug, and eye drops can be delivered from a disposable single-use container or a multi-use container. Regardless of the type of use, sterility of the formulation is important. While some formulations can be terminally sterilized in a container by, for example, autoclaving, other formulations are not amenable to such process due to thermal instability of the active ingredient. To avoid thermal stability issues, formulations can also be filter sterilized. However, where the formulation contains a viscosity modifier, filter sterilization may be unsuitable as well.

In still further cases, one or more ingredients may not be heat stable and the formulation may include viscosity enhancers that render sterile filtration impracticable or even impossible. For example, U.S. Pat. No. 11,071,732 teaches ophthalmic compositions that are prepared by autoclaving one portion of a formulation (containing a viscosity modifier) and filter sterilizing another portion of the formulation (containing low dose atropine), and then combining both portions to so form a sterile composition that can then be packaged into a sterile container.

In general, numerous methods of sterilizing a container are known in the art, including sterilization using gamma radiation (e.g., at 25 kGy), or autoclaving at 121° C. for 15 about minutes, or sterilization with ethylene oxide gas, or heating with a bactericide at 98° C. to 100° C. for about 30 minutes. However, it is not known in the art, if and to what extent, the sterilization process will affect one or more ingredients of a formulation, particularly where one or more ingredients of the formulation are prone to degradation. In addition, the presence of leachables in various medical devices is well known and may lead to accumulation of undesirable components in the liquid contained in the container. In addition, leachables may also react with an active ingredient in the liquid contained in the container to so reduce the concentration of active ingredients. Reduction of leachables can be achieved with heat treatment under reduced pressure as described in US 2011/0190711. However, it is once more generally not known whether sterilization processes have significant impact on the quantity of leachables in a container, especially for multi-dose containers where the composition stored in the container is exposed to the container over an extended period of time.

Difficulties associated with leachables may also extend to interactions with inactive ingredients, and particularly with viscosity modifiers where the leachables and/or impurities can lead to partial degradation of the viscosity modifiers. Such degradation will then lead to a drop in viscosity with concomitant loss in intended function. The presence of leachables and inadvertent change in viscosity is particularly problematic where the fluid contained in the multi-dose container is an eye drop formulation that contains a labile active ingredient. In such case, the leachables and inadvertent change in viscosity will led to significant loss of active ingredient and therapeutic effect that is further compounded by increased runoff from the eye due to the decreased viscosity. Leachable peaks appearing in the chromatographic window could also interfere with identification of drug product impurities and can make quantitative analysis challenging.

Thus, even though various packaging compositions and methods for ophthalmic formulations are known in the art, all or almost all of them suffer from several drawbacks. Therefore, there remains a need for improved packaging compositions and methods for ophthalmic formulations, especially as it relates to low-dose atropine ophthalmic compositions in a multi-use container.

### SUMMARY OF THE INVENTION

The inventive subject matter is directed to various kits, compositions, and methods of multi-dose ophthalmic consumer products and methods therefor where the sterile ophthalmic composition in the product has desirable storage stability, maintains viscosity and low content of total impurities, even when stored over extended periods. Such kits, compositions, and methods are especially desirable for use with low-dose atropine eye drops that are stored in a multi-dose container.

In one aspect of the inventive subject matter, the inventors contemplate a method of manufacturing a multi-dose ophthalmic consumer product that includes a step of providing a sterile ophthalmic composition comprising a therapeutic agent and a viscosity modifier that generates a dynamic viscosity of between 5 and 50 cP (centipoise). In another step, the sterile ophthalmic composition is filled into a sterilized container, wherein the container is prepared from a polymer and is sterilized in a process that, post-sterilization and after storage of the ophthalmic composition at 40° C. for at least 6 months, limits (a) loss of dynamic viscosity to equal or less than 5 cP, and (b) total impurities leached from the container to equal or less than 6.5 wt %.

In some embodiments, the therapeutic agent is atropine or a pharmaceutically acceptable salt thereof, which may be present at relatively low concentrations (e.g., equal or less than 0.05 wt %). In further embodiments, the viscosity modifier is a cellulosic viscosity modifier such as a hydroxyethyl cellulose, a hydroxypropyl cellulose, and/or a hydroxypropyl methylcellulose (which may or may not be chemically modified). Alternatively, the viscosity modifier may also be a non-cellulosic viscosity modifier (e.g., a polymeric compound, a polysaccharidic polymer, or glycerol).

In still further embodiments, the sterile ophthalmic composition may be prepared by combining a filter sterilized first solution containing the therapeutic agent and an autoclaved second solution containing the viscosity modifier. In some examples, the sterile ophthalmic composition has a dynamic viscosity of between 10 and 40 cP, or between 10 and 30 cP. Most typically, the step of filling will comprise aseptic filling. In yet further embodiments, the polymer may be polypropylene or low-density polyethylene, and/or the sterilization process comprises gaseous sterilization (e.g., ethylene oxide sterilization).

In some aspects, the loss of dynamic viscosity is equal or less than 4 cP, or less than 2.5 cP, or less than 1.0 cP, and/or the total impurities leached from the container are equal or less than 4.0 wt %, or equal or less than 2.5 wt %, or equal or less than 1.5 wt %.

Consequently, the inventors also contemplate a multi-dose ophthalmic consumer product that comprises a container enclosing a sterile ophthalmic composition that includes a therapeutic agent and a viscosity modifier in an amount sufficient to generate a dynamic viscosity of the ophthalmic composition between 5 and 50 cP (centipoise). Most typically, the container is a sterile polymeric container, and the ophthalmic composition, after storage of the ophthalmic composition at 40° C. for at least 6 months, has (a) a loss of dynamic viscosity of equal or less than 5 cP, and (b) total impurities leached from the container in an amount of equal or less than 6.5 wt %.

With respect to the therapeutic agent, the viscosity modifier, the dynamic viscosity, and the polymer for the container, the loss of viscosity, and the total impurities leached from the container, the same considerations as provided above apply.

Various objects, features, aspects and advantages of the inventive subject matter will become more apparent from the following detailed description of preferred embodiments, along with the accompanying drawing figures in which like numerals represent like components.

### DETAILED DESCRIPTION

The inventors have unexpectedly discovered that the type of container material and type of sterilization method for the container has significant impact on various parameters of a composition stored in the container, particularly where the composition is stored in the container over an extended period of time and includes an active ingredient and/or viscosity modifier that is/are sensitive to degradation.

For example, and as is described in more detail below, it was observed that certain liquid low-dose atropine formulations for ophthalmic topical administration had significant quantities of total impurities for leachables, significant loss in viscosity, and significant presence of atropine related degradation products when the low-dose atropine formulation was stored in a commonly used LDPE (low density polyethylene) container that was subjected to gamma radiation for sterilization. In contrast, the same formulation stored for the same time under the same conditions unexpectedly exhibited substantially lower quantities of total impurities for leachables, only a minor loss in viscosity, and no detectable atropine related degradation products when the low-dose atropine formulation was stored in an LDPE container that was subjected to ethylene oxide for sterilization. Equally remarkably, even lower quantities of total impurities for leachables, near negligible loss in viscosity, and no detectable atropine related degradation products were observed when the low-dose atropine formulation was stored under the same conditions and duration in a PP (polypropylene) container that was subjected to ethylene oxide for sterilization as is described in more detail below.

Therefore, especially preferred therapeutic agents are atropine or a pharmaceutically acceptable salt thereof in an aqueous solution, typically at a concentration of equal or less than 0.05 wt %. Moreover, it is typically preferred that the viscosity modifier is a cellulosic viscosity modifier (e.g., hydroxyethyl cellulose, hydroxypropyl cellulose, and hydroxypropyl methylcellulose, each of which may be further substituted) or a non-cellulosic viscosity modifier (e.g., a polymeric compound, a polysaccharidic polymer, or glycerol). Most typically, but not necessarily, it is contemplated that the ophthalmic composition has a dynamic viscosity of between 10 and 40 cP or between 10 and 30 cP. The ophthalmic composition will preferably also include a viscosity modifier that helps generate a dynamic viscosity of the ophthalmic composition between 5 and 50 cP (centipoise).

Exemplary formulations and their methods of manufacture are suitable for use herein include those described in U.S. Ser. No. 10/251,875, U.S. Ser. No. 10/583,132, U.S. Ser. No. 10/576,074, U.S. Ser. No. 10/610,525, U.S. Ser. No. 11/071,732, and U.S. Ser. No. 10/568,875, and those in US 2020/0352928, US 2020/0405705, US 2020/0397775, US 2020/0397776, and US 2021/0128546, all of which are incorporated by reference herein.

In view of the above and further experimental data provided below, the ophthalmic composition will have, after storage of the ophthalmic composition at 40° C. for at least 6 months in the container a loss of dynamic viscosity of equal or less than 5 cP (or equal or less than 4 cP, or equal or less than 2.5 cP, or equal or less than 1.0 cP), and total impurities leached from the container in an amount of equal or less than 6.5 wt % (or equal or less than 5.5 wt %, or equal or less than 4.5 wt %, or equal or less than 3.5 wt %, or equal or less than 2.5 wt %, or equal or less than 1.5 wt %). Moreover, the ophthalmic composition will have, after storage of the ophthalmic composition at 40° C. for at least 6 months in the container a loss of atropine of equal or less than 5% (or equal or less than 4%, or equal or less than 3%, or equal or less than 2%, or equal or less than 1%). Viewed from a different perspective, the stability and purity of pharmaceutical composition can be maintained over extended periods using the systems and methods presented herein.

With respect to suitable containers, it is contemplated that all container materials are deemed suitable for use herein, including containers manufactured from glass, or from one or more polymeric materials, so long as such containers will have upon sterilization no or only minimal impact on various parameters of the composition stored in the container as described in more detail below. For example, suitable container materials include polypropylene, polystyrenes, polyethylene, polyethylene terephthalate, poly(vinyl chloride), polyamides, Teflon, high-density polyethylene (HDPE), low-density polyethylene (LDPE), polycarbonates, polycyanoacrylates, poly(vinyl acetates), cyclic olefin copolymers (COC), and any copolymers thereof. Moreover, it is contemplated that where the container has a closure system, contemplated closure systems can be made from the same polymer or a different polymer.

It is further contemplated that suitable container volumes and configurations will include all container configurations and volumes. However, especially preferred configurations are consumer products and particularly eye drop containers in single-dose and multi-dose format. Therefore, especially contemplated containers include multi-use containers having internal volumes of between 1 mL and 100 mL, and most preferably between 5 and 20 mL. Moreover, preferred containers will include a closure mechanism that allows temporary covering of the container opening (e.g., twist cap, screw cap, snap lid, etc.). It is also preferred that the containers are configured to dispense individual drops as is common with eye drop containers. In particularly preferred aspects, the containers will include an internal mechanism that allows dispensing of single drops while maintaining sterility of the container content. For example, containers may be configured to dispense drops having a volume of between 10 and 100 μL, and more preferably between 20 and 60 μL (e.g., 40 μL). Moreover, the container tips will preferably be configured to allow dropwise delivery of the pharmaceutical composition where the composition has a dynamic viscosity of between about 10-200 cP (e.g., viscosity between 10 and 30 cP, or between 20 and 40 cP, or between 30 and 50 cP, or between 40 and 100 cP).

Therefore, in one aspect of the inventive subject matter, a sterile ophthalmic composition is aseptically filled into a sterile polymeric container, that is most typically configured as a multi-dose ophthalmic eye drop container to so form an ophthalmic consumer product.

Preferably, as also described in more detail below, the polymer container is manufactured from polypropylene or low-density polyethylene, has a multi-use eye dropper format, and will contain between 1 and 50 mL, and more typically between 1 and 20 mL of the pharmaceutical composition. Furthermore, it is preferred that the sterile polymeric container is a gas-sterilized (e.g., using ethylene oxide). Likewise, numerous methods of sterilization of the container are contemplated herein and will be deemed suitable for use herein so long as they have no or only minimal impact on various parameters of the composition stored in the container. Suitable sterilization methods include autoclaving, gaseous sterilization with one or more volatile compounds, e-beam radiation, X-ray radiation, gamma radiation, thermal sterilization with antimicrobial agents, etc. Of course, it should be appreciated that these changes in compositional parameters may be due to the container material per se or due to the container material reaction with a sterilizing condition such a specific container material when subjected to sterilizing conditions by autoclaving, gamma radiation, gaseous sterilization, etc.

For example, reduction in concentration or activity of the active pharmaceutical agent can be measured by standardized HPLC, MS, biochemical or biological assays, and the particular nature of the active pharmaceutical agent will at least in part determine the type of assay used. Of course, it should be appreciated that the reduction in concentration or activity of the active pharmaceutical agent can be due to one or more factors, including an increase in one or more degradation products of the active pharmaceutical agent, for example, via oxidative degradation, polymerization or aggregation, adduct formation, isomerization, etc. Such increase in one or more degradation products (e.g., tropic acid) of the active pharmaceutical agent (e.g., atropine) is preferably less than 10 wt %, or less than 8 wt %, or less than 6 wt %, or less than 5 wt %, or less than 4 wt %, or less than 3 wt %, or less than 2 wt %, or less than 1 wt %, or less than 0.5 wt %, or less than 0.3 wt %, over a period of at least 6 months when the composition is stored in the container at 40° C.

Likewise, reduction in concentration or activity of an ingredient other than the active pharmaceutical agent, such as antioxidant loss, viscosity loss, loss in chelation ability, phase separation, aggregation of non-API, change in pH, etc. is preferably also maintained to a minor level For example, where the reduction in concentration or activity of an ingredient is loss of an antioxidant, the less is preferably less than less than 20%, or less than 15%, or less than 10%, or less than 8%, or less than 6%, or less than 4%, or less than 2%, or less than 1%, or less than 0.5t % over a period of at least 6 months when the composition is stored in the container at 40° C. In another example, where the reduction in concentration or activity of an ingredient is viscosity loss, the loss of viscosity is preferably less than 10 cP, or less than 8 cP, or less than 6 cP, or less than 5 cP, or less than 4 cP, or less than 3 cP, or less than 2 cP, or less than 1 cP over a period of at least 6 months when the composition is stored in the container at 40° C.

Likewise, where the reduction in concentration or activity of an ingredient is loss in chelation ability, the loss is preferably less than 15% in available chelator, or less than 10% in available chelator, less than 8% in available chelator, less than 6% in available chelator, less than 4% in available chelator, less than 3% in available chelator, less than 2% in available chelator over a period of at least 6 months when the composition is stored in the container at 40° C. In still further examples, where the reduction in concentration or activity of an ingredient is a change in pH, the change in pH is preferably less than 0.5 pH units, less than 0.4 pH units, less than 0.3 pH units, less than 0.2 pH units, less than 0.1 pH units over a period of at least 6 months when the composition is stored in the container at 40° C.

### EXAMPLES

The following examples illustrate some of the experiments leading to the formulations according to the inventive subject matter, however, should not be construed to limit the scope of the claims in any way.

Quantitative Analyses: A combined test method based on Ultra Performance Liquid Chromatography (UPLC) was developed to perform identification, assay and determination of related compounds in a single run. This was accomplished by using a reversed-phase gradient UPLC with the UV detection including on-line acquisition of UV absorption spectra. Octadecylsilyl-functionalized silica with sub-2 μm particles was used as a stationary phase for chromatographic analysis. The mobile phase is prepared by mixing an aqueous buffer solution with an acidic pH and an acetonitrile-water mixture. Quantification of the active ingredient and related compounds is performed by comparing corresponding peak responses from a Sample Solution to the atropine peak response from a Standard solution. Relative response factors are used to correct for chemical structure effects on the responses. Two identification methods are incorporated into this test method. Atropine is identified based on the retention time of the major peak in the Sample Solution chromatogram and on the UV absorption spectrum acquired within this peak.

Exemplary Formulations And Stability Tests: Ophthalmic ready-to-use low-dose atropine formulations were prepared using a two-step process. Step 1—Preparation of the Polymer Solution Phase: To about 60% of WFI the required quantity of HPMC was added slowly and mixed until a clear solution was observed. The solution was then subjected to autoclaving at 121° C. for a period of about 30 min. Step 2—Preparation of the Drug Solution Phase: To about 30% of WFI the required quantities of disodium edetate, monobasic sodium phosphate, dibasic sodium phosphate and sodium chloride were added sequentially upon complete dissolution of each ingredient. The pH of the solution was measured and adjusted to about 5.5±0.1 using hydrochloric acid/sodium hydroxide. To the above solution atropine sulfate was added and mixed until there was complete dissolution. The Drug Solution from Step 2 was then mixed with the Polymer Solution in Step 1. The batch volume was made up using WFI to yield the pharmaceutical composition. Tables 1-3 below provide exemplary formulations used for the stability studies. Unless otherwise indicated, pharmaceutical compositions of Table 3 (50 mM Buffer Composition with NaCl, low EDTA) were subjected to long term stability studies using 0.01 wt % atropine sulfate.

TABLE 1

| | | | | 100 mM Buffer Composition | |
|---|---|---|---|---|---|
| | | 100 mM Buffer Composition | | (Low EDTA) | |
| No. | Ingredient | % w/v | % w/v | % w/v | % w/v |
| 1 | Atropine Sulfate | 0.01 or 0.02 | 0.01 or 0.02 | 0.01 or 0.02 | 0.01 or 0.02 |
| 2 | Sodium Dihydrogen | 0.059 | 0.06 | 0.059 | 0.06 |
| | Phosphate Anhydrous | | | | |
| 3 | Disodium Hydrogen | 1.15 | 1.16 | 1.15 | 1.16 |
| | Phosphate Anhydrous | | | | |
| 4 | Edetate Sodium | 0.10 | 0.10 | 0.01 | 0.01 |
| 5 | Sodium Chloride | — | — | — | — |
| 6 | Hypromellose 2910 | 0.50 | 0.50 | 0.50 | 0.50 |
| | (Benecel ™ E4M Pharm1) | | | | |

| | | | |
|---|---|---|---|
| 7 | Hydrochloric Acid | Q.S. for pH adjustment | Q.S. for pH adjustment |
| 8 | Sodium Hydroxide | Q.S. for pH adjustment | Q.S. for pH adjustment |
| 9 | Water for Injection | Q.S. to 100% | Q.S. to 100% |

TABLE 2

| | | 75 mM Buffer Composition | | No Buffer Composition | |
|---|---|---|---|---|---|
| | | with NaCl | | With NaCl | |
| No. | Ingredient | % w/v | % w/v | % w/v | % w/v |
| 1 | Atropine Sulfate | 0.01 or 0.02 | 0.01 or 0.02 | 0.01 or 0.02 | 0.01 or 0.02 |
| 2 | Sodium Dihydrogen | 0.044 | 0.04 | — | — |
| | Phosphate Anhydrous | | | | |
| 3 | Disodium Hydrogen | 0.863 | 0.87 | — | — |
| | Phosphate Anhydrous | | | | |
| 4 | Edetate Sodium | 0.1 | 0.1 | 0.1 | 0.1 |
| 5 | Sodium Chloride | 0.15 | 0.15 | 0.9 | 0.91 |
| 6 | Hypromellose 2910 | 0.5 | 0.5 | 0.5 | 0.5 |
| | (Benecel ™ E4M Pharm1) | | | | |

| | | | |
|---|---|---|---|
| 7 | Hydrochloric Acid | Q.S. for pH adjustment | Q.S. for pH adjustment |
| 8 | Sodium Hydroxide | Q.S. for pH adjustment | Q.S. for pH adjustment |
| 9 | Water for Injection | Q.S. to 100% | Q.S. to 100% |

TABLE 3

| | | 50 mM Buffer Composition | | 50 mM Buffer Composition | |
|---|---|---|---|---|---|
| | | with NaCl | | with NaCl, low EDTA | |
| No. | Ingredient | % w/v | % w/v | % w/v | % w/v |
| 1 | Atropine Sulfate | 0.01 or 0.02 | 0.01 or 0.02 | 0.01 or 0.02 | 0.01 or 0.02 |
| 2 | Sodium Dihydrogen | 0.0295 | 0.03 | 0.0295 | 0.03 |
| | Phosphate Anhydrous | | | | |
| 3 | Disodium Hydrogen | 0.575 | 0.58 | 0.575 | 0.58 |
| | Phosphate Anhydrous | | | | |
| 4 | Edetate Sodium | 0.1 | 0.1 | 0.01 | 0.01 |
| 5 | Sodium Chloride | 0.25 | 0.25 | 0.25 | 0.25 |
| 6 | Hypromellose 2910 | 0.5 | 0.5 | 0.5 | 0.5 |
| | (Benecel ™ E4M Pharm1) | | | | |

| | | | |
|---|---|---|---|
| 7 | Hydrochloric Acid | Q.S. for pH adjustment | Q.S. for pH adjustment |
| 8 | Sodium Hydroxide | Q.S. for pH adjustment | Q.S. for pH adjustment |
| 9 | Water for Injection | Q.S. to 100% | Q.S. to 100% |

Of course, it should be appreciated that the above formulations are exemplary only, and that various modification can be made as already discussed above, including omission of a buffer and/or chelating agent, replacement of NaCl with other tonicity agents, and/or use of viscosity modifiers other than Hypromellose 2910.

To investigate influence of the container material and manner of sterilization on one or more parameters of the ophthalmic composition, the inventors subjected test formulations to extended storage stability assays under ambient storage conditions (25° C.) and accelerated storage conditions (40° C.) using test formulations and placebo formulations (i.e., as test formulations, but without atropine). Exemplary results are shown in Tables 4-7 below.

As can be readily seen form the results in Table 4, the polypropylene and low-density polyethylene containers performed similarly without sterilization (NS) with regard to pH stability, showing only a slight acidification. Sterilization with ethylene oxide (ETO) and gamma irradiation (Gamma) had no apparent effect on pH stability/drift. On the other hand, viscosity changed mildly over 6 months upon sterilization with ETO but had a significant drop when gamma sterilization was used. Moreover, gamma sterilization also provided the highest level and number of total impurities, suggesting that gamma radiation produces significant leachable contaminants. In contrast, when ETO was used, the number and total quantity of as placebo peaks or unknown peaks observed in placebo was minimal for sterilized polypropylene containers, and moderate for sterilized LDPE containers.

TABLE 4

| | | Atropine Study, 0.01%, PLACEBO; Multi-dose Configuration Stability | | | | | | | |
|---|---|---|---|---|---|---|---|---|---|
| | | 40° C. | | | | | | | |
| | | 1 Month | | | | | 3 months | | |
| | | NS | ETO | NS | ETO | Gamma | NS | ETO | NS |
| Lot: 12850 | | PP | PP | LDPE | LDPE | LDPE | PP | PP | LDPE |
| Appearance | | CCS | CCS | CCS | CCS | CCS | CCS | CCS | CCS |
| Viscosity | | 20.56 | 21.17 | 16.71 | 19.73 | 10.33 | NT | NT | NT |
| pH | | 5.26 | 5.28 | 5.21 | 5.27 | 5.21 | 5.15 | 5.14 | 5.14 |
| Assay (%) | | ND | ND | ND | ND | ND | ND | ND | ND |
| Related | Unknown | 0.06 | 0.07 | 0.07 | 0.07 | 0.14 | 0.08 | NR | 0.14 |
| Compounds | Impurities | 0.1 | 0.1 | 0.10 | 0.09 | 0.09 | 0.05 | 0.05 | 0.12 |
| (%) | | 0.05 | 0.05 | 0.05 | 0.06 | 0.09 | NR | NR | 0.26 |
| | | 0.29 | 0.17 | 1.39 | 1.27 | 1.69 | | | 0.20 |
| | | NR | NR | NR | NR | NR | | | 0.13 |

| | | | | | | | | |
|---|---|---|---|---|---|---|---|---|
| Total | 0.50 | 0.39 | 1.61 | 1.49 | 2.09 | 0.13 | 0.05 | 0.85 |

| | | Atropine Study, 0.01%, PLACEBO; Multi-dose Configuration Stability | | | | | | |
|---|---|---|---|---|---|---|---|---|
| | | 40° C. | | | | | | |
| | | 3 months | | 6 Months | | | | |
| | | ETO | Gamma | NS | ETO | NS | ETO | Gamma |
| Lot: 12850 | | LDPE | LDPE | PP | PP | LDPE | LDPE | LDPE |
| Appearance | | CCS | CCS | CCS | CCS | CCS | CCS | CCS |
| Viscosity | | NT | NT | 18.07 | 19.30 | 12.30 | 15.69 | 5.99 |
| pH | | 5.17 | 5.18 | 5.09 | 5.12 | 5.10 | 5.13 | 5.12 |
| Assay (%) | | ND | ND | ND | ND | ND | ND | ND |
| Related | Unknown | 0.06 | 0.10 | NR | NR | NR | NR | 0.25 |
| Compounds | Impurities | NR | 0.12 | 1.58 | 1.67 | 4.69 | 5.69 | 5.98 |
| (%) | | 0.19 | 0.17 | NR | NR | 0.29 | 0.23 | 0.35 |
| | | 0.28 | 0.20 | | | 0.24 | 0.28 | 0.36 |
| | | NR | 0.10 | | | | | 0.16 |

| | | | | | | | |
|---|---|---|---|---|---|---|---|
| Total | 0.53 | 0.69 | 1.58 | 1.63 | 5.22 | 6.20 | 7.10 |

ND—Impurities not detected;
NR—Impurities over 0.01 but less than 0.05%

Similarly, when ophthalmic compositions included atropine, pH and viscosity changes were similar to placebo control. Once more, with regard to quantity and total number of impurities not attributable to atropine, sterilized PP containers performed better than sterilized LDPE containers, while gamma radiated LDPE containers had the highest level and number of impurities. In addition, gamma irradiated containers also afforded the highest loss of viscosity and quantity and number of degradation products of atropine as can be seen from the data in Table 5 and Table 6.

TABLE 5

| | | NVK002-DRUG PRODUCT, 0.01%, | | | | |
|---|---|---|---|---|---|---|
| | | Multi-dose Configuration Stability | | | | |
| | | 25° C. | | | | |
| | | 6 Months (Novelia) | | | | |
| | | NS | ETO | NS | ETO | |
| Lot: 12850 | | PP | PP | LDPE | LDPE | GammaLDPE |
| Appearance | | CCS | CCS | CCS | CCS | CCS |
| Viscosity | | 22.75 | 22.61 | 17.54 | 22.02 | 14.02 |
| pH | | 5.16 | 5.13 | 5.13 | 5.16 | 5.16 |
| Assay (%) | | 101.7 | 101.5 | 101.4 | 101.9 | 101.5 |
| Related | DL - Tropic Acid | 0.54 | 0.54 | 0.53 | 0.54 | 0.53 |
| Compounds | Apoatropine | NR | 0.03 | NR | 0.04 | NR |
| (%) | C-C system peaks(Placebo) | 0.07 | — | 0.27 | 0.28 | 0.09 |
| | | | | 0.07 | 0.06 | 0.34 |
| | Unknown Impurities (RRT) | — | — | — | — | — |

| | | | | | |
|---|---|---|---|---|---|
| Total | 0.61 | 0.57 | 0.87 | 0.92 | 0.96 |

ND—Impurities not detected;
NR—Impurities less than 0.05%

TABLE 6

| | | NVK002-DRUG PRODUCT, 0.01%, | | | | |
|---|---|---|---|---|---|---|
| | | Multi-dose Configuration Stability | | | | |
| | | 40° C. | | | | |
| | | 6 Months | | | | |
| | | NS | ETO | NS | ETO | Gamma |
| Lot: 12850 | | PP | PP | LDPE | LDPE | LDPE |
| Appearance | | CCS | CCS | CCS | CCS | CCS |
| Viscosity | | 19.03 | 19.28 | 14.95 | 18.87 | 6.14 |
| pH | | 5.18 | 5.16 | 5.16 | 5.14 | 5.14 |
| Assay (%) | | 97.8 | 97.6 | 97.8 | 98.1 | 97.3 |
| Related | DL - Tropic Acid | 2.65 | 2.66 | 2.65 | 2.67 | 2.25 |
| Compounds | Apoatropine | 0.21 | 0.21 | 0.20 | 0.20 | 0.18 |
| (%) | C-C system peaks | 1.66 | 1.20 | 5.05 | 4.65 | 0.12 |
| | (Placebo) | | | | 0.18 | 4.31 |
| | | | | | | 0.17 |
| | | | | | | 0.20 |
| | Unknown | — | — | — | — | 0.16_{(0.27)} |
| | Impurities_{(RRT)} | | | | | 0.08_{(1.20)} |
| | | | | | | 0.26_{(1.10)} |

| | | | | | |
|---|---|---|---|---|---|
| Total | 4.52 | 4.07 | 4.99 | 7.70 | 7.73 |

ND—Impurities not detected;
NR—Impurities less than 0.05%

Therefore, the inventors also contemplate a multi-dose ophthalmic consumer product that includes a container enclosing a sterile ophthalmic composition. The sterile ophthalmic composition preferably includes atropine or atropine sulfate at a concentration of between about 0.01% and 0.02%, and a cellulosic viscosity modifier in an amount that generates a dynamic viscosity of the ophthalmic composition between 5 and 50 cP (centipoise), and more preferably between 15-25 cP. I especially contemplated aspects, the container is an ethylene oxide sterilized polypropylene or low density polyethylene container that has a volume between 1 and 20 mL, and that is configured as a multi-dose container (e.g., including a one-way valve to maintain sterility). Most typically, the container will also be configured such that each drop will have a volume of about 20-60 microliter. Especially contemplated cellulosic viscosity modifiers include hydroxypropyl methylcellulose, and where desired, the ophthalmic composition may further include a low-strength phosphate buffer (e.g., strength of equal or less than 75 mM). Moreover, contemplated ophthalmic formulations may include additional agents such as a tonicity agent (e.g., NaCl) and/or a chelator (e.g., EDTA).

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A method of manufacturing a multi-dose ophthalmic consumer product, comprising:
providing a sterile ophthalmic composition comprising a therapeutic agent, wherein the therapeutic agent is atropine or a pharmaceutically acceptable salt thereof, and further comprising a viscosity modifier that generates a dynamic viscosity of between 5 and 50 cP (centipoise);
aseptically filling the sterile ophthalmic composition into a sterilized container;
wherein the container is prepared from a polymer and is sterilized in a process that post-sterilization and after storage of the ophthalmic composition at 40° C. for at least 6 months
(a) limits loss of dynamic viscosity to equal or less than 5 cP, and
(b) limits total impurities leached from the container to equal or less than 6.5 wt % as determined by reverse phase UPLC.

2. (canceled)

3. The method of claim 1 wherein the therapeutic agent present in the ophthalmic composition at a concentration of equal or less than 0.05 wt %.

4. The method of claim 1 wherein the viscosity modifier is a cellulosic viscosity modifier.

5. The method of claim 4 wherein the cellulosic viscosity modifier is a hydroxyethyl cellulose, a hydroxypropyl cellulose, and a hydroxypropyl methylcellulose.

6. The method of claim 1 wherein the viscosity modifier is a non-cellulosic viscosity modifier.

7. The method of claim 6 wherein the non-cellulosic viscosity modifier is a polymeric compound, a polysaccharidic polymer, or glycerol.

8. The method of claim 1 wherein the sterile ophthalmic composition is prepared by combining a filter sterilized first solution containing the therapeutic agent and an autoclaved second solution.

9. The method of claim 1 the sterile ophthalmic composition has a dynamic viscosity of between 10 and 40 cP.

10-11. (canceled)

12. The method of claim 1 wherein the polymer is polypropylene, a cyclic olefin copolymer, a cyclic olefin polymer, or low-density polyethylene.

13. The method of claim 1 wherein the sterilization process comprises gaseous sterilization.

14. (canceled)

15. The method of claim 1 wherein the loss of dynamic viscosity is equal or less than 4 cP, and/or wherein total impurities leached from the container are equal or less than 4.0 wt %.

16-20. (canceled)

21. A multi-dose ophthalmic consumer product, comprising:
a container enclosing a sterile ophthalmic composition that includes a therapeutic agent, wherein the therapeutic agent is atropine or a pharmaceutically acceptable salt thereof, and a viscosity modifier in an amount sufficient to generate a dynamic viscosity of the ophthalmic composition between 5 and 50 cP (centipoise);
wherein the container is a sterile polymeric container; and
wherein the ophthalmic composition, after storage of the ophthalmic composition at 40° C. for at least 6 months, has
(a) a loss of dynamic viscosity of equal or less than 5 cP, and
(b) total impurities leached from the container in an amount of equal or less than 6.5 wt % as determined by reverse phase UPLC.

22. (canceled)

23. The ophthalmic consumer product of claim 21 wherein the therapeutic agent present in the ophthalmic composition at a concentration of equal or less than 0.05 wt %.

24. The ophthalmic consumer product of claim 21 wherein the viscosity modifier is a cellulosic viscosity modifier.

25. The ophthalmic consumer product of claim 24 wherein the cellulosic viscosity modifier is a hydroxyethyl cellulose, a hydroxypropyl cellulose, and a hydroxypropyl methylcellulose.

26. The ophthalmic consumer product of claim 21 wherein the viscosity modifier is a non-cellulosic viscosity modifier.

27. The ophthalmic consumer product of claim 26 wherein the non-cellulosic viscosity modifier is a polymeric compound, a polysaccharidic polymer, or glycerol.

28-31. (canceled)

32. The ophthalmic consumer product of claim 21 wherein the polymer is polypropylene, a cyclic olefin copolymer, a cyclic olefin polymer, or low-density polyethylene, and wherein the sterile polymeric container is an ethylene oxide-sterilized container.

33-40. (canceled)

41. A multi-dose ophthalmic consumer product, comprising:
a container enclosing a sterile ophthalmic composition that includes atropine or atropine sulfate at a concentration of between about 0.01% and 0.02%, and a cellulosic viscosity modifier in an amount that generates a dynamic viscosity of the ophthalmic composition between 5 and 50 cP (centipoise);
wherein the container is an ethylene oxide sterilized container made from polypropylene, a cyclic olefin copolymer, a cyclic olefin polymer, or low density polyethylene container and has a volume between 1 and 20 mL; and
wherein the container is configured as a multi-dose container.

42. The ophthalmic consumer product of claim 41, wherein the cellulosic viscosity modifier is a hydroxypropyl methylcellulose, wherein the ophthalmic composition further includes a phosphate buffer at a strength of equal or less than 75 mM, and/or wherein the container is an ethylene oxide sterilized polypropylene container.

43-44. (canceled)
